(19) United States
(12) Patent Application Publication
(10) Pub. No.: US 2023/0090683 A1
(43) Pub. Date: Mar. 23, 2023

(54) ASPACYTARABINE PHARMACEUTICAL COMPOSITIONS AND USES THEREOF

(71) Applicant: Biosight Ltd., Lod (IL)

(72) Inventors: Margarita SHUMILOV, Herzliya (IL); Shoshanna TESSLER, Zichron Yaakov (IL)

(73) Assignee: Biosight Ltd., Lod (IL)

(21) Appl. No.: 17/797,443

(22) PCT Filed: Feb. 4, 2021
(86) PCT No.: PCT/IL2021/050137

§ 371 Date: Aug. 4, 2022

Related U.S. Application Data
(60) Provisional application No. 62/969,769, filed on Feb. 4, 2020.

(51) Int. Cl. A61K 31/7068; A61K 47/10; A61K 47/32; A61K 47/34; A61K 47/40

(52) U.S. Cl. CPC A61K 31/7068; A61K 47/10; A61K 47/32; A61K 47/34; A61K 47/40

(57) ABSTRACT

The present invention relates to a composition comprising aspacytarabine (also known as BST-236 and Astarabine®) or a pharmaceutically acceptable salt thereof and an stabilizer and/or solubilizer, wherein the stabilizer and/or solubilizer is selected from at least one water soluble stabilizer and/or solubilizer such as a linear polymer or derivative thereof, an inclusion compound (i.e. cyclodextrin) and combination thereof. The present invention further relates to uses of the composition, particularly for use in the treatment of neoplastic disease.

### FIELD OF THE INVENTION

The present invention relates to a pharmaceutical composition comprising (S)-2-amino-4-((1-(( 2R,3S,4S,5R)-3,4-dihydroxy-5-(hydroxymethyl)tetrahydrofuran-2-yl )-2-oxo-1,2-dihydropyrimidin-4-yl)amino)-4-oxobutanoic acid (also known as BST-236, Astarabine® or aspacytarabine) or a pharmaceutically acceptable salt thereof and at least one water soluble stabilizer and/or solubilizer, wherein the at least one water soluble stabilizer and/or solubilizer is selected from a linear polymer, an inclusion compound (i.e. cyclodextrin) and combination thereof. The present invention further relates to the use of the composition, particularly for use in the treatment of neoplastic diseases.

### BACKGROUND OF THE INVENTION

The use of prodrugs to impart desired characteristics such as bioavailability, pharmacokinetics, or increased site-specificity is a recognized concept in the art of pharmaceutical development. For example, direct or indirect conjugation of a drug to an antibody creates a stable conjugate that can arrive at the target site with minimum dissociation of the drug. Drug targeting may be combined with a mechanism of selective release of the drug for maximal potency.

International Patent Application Publication No. WO/2017/093993 teaches prodrugs comprising cytarabine conjugated to a single amino acid selected from the group consisting of aspartic acid, glutamic acid, asparagine, and glutamine, for use in treating neoplastic diseases in medically compromised subjects. Specifically, this application teaches a method where aspacytarabine, which is a conjugate of cytarabine and aspartic acid wherein cytarabine is covalently attached to the carboxyl group of the side chain of aspartic acid, is administered to patients having acute lymphocytic leukemia (ALL), acute myeloid leukemia (AML), or myelodysplastic syndrome (MDS), thereby prolonging the survival of these patients.

The inventors of the present invention have found that the aspacytarabine, having the amino, and carboxylic acid groups with a neutral charge at the isoelectric point, (“free base compound”) has low solubility in aqueous media, while the aspacytarabine acid salt which is soluble in aqueous media is not stable and decomposes over time. Thus, there is an unmet need for a stable and highly concentrated solutions and/or high dosage composition comprising aspacytarabine.

### SUMMARY OF THE INVENTION

According to one aspect, this invention provides a composition comprising (S)-2-amino-4-(( 1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (aspacytarabine) or pharmaceutically acceptable salt thereof and at least one water soluble stabilizer and/or solubilizer.

According to one aspect this invention provides a composition comprising (S)-2-amino-4-(( 1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (aspacytarabine) or pharmaceutically acceptable salt thereof and at least one water soluble stabilizer and/or solubilizer; wherein the composition is chemically and physically stable when stored at a temperature between -80 to 30° C. for at least 1 month and for at least 24 hrs when formulated as an aqueous solution.

According to one aspect this invention provides a composition comprising (S)-2-amino-4-(( 1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (aspacytarabine) or pharmaceutically acceptable salt thereof and at least one water soluble stabilizer and/or solubilizer wherein the concentration of aspacytarabine within the composition in an aqueous solution is between 20 -1000 mg/ml of aspacytarabine or pharmaceutically acceptable salt thereof.

According to another aspect of this invention, the at least one water soluble stabilizer and/or solubilizer is selected from a linear polymer, an inclusion compound (i.e. cyclodextrin) and combination thereof.

In one further aspect, the present invention provides a method of treating a neoplastic disease comprising administering to a subject in need of such treatment a pharmaceutical composition comprising (S)-2-amino-4-((1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (aspacytarabine) or a pharmaceutically acceptable salt thereof and at least one water soluble stabilizer and/or solubilizer. In another aspect of this invention, the at least one water soluble stabilizer and/or solubilizer is selected from a linear polymer, cyclodextrin and combination thereof.

### BRIEF DESCRIPTION OF THE FIGURES

The subject matter regarded as the present invention is particularly pointed out and distinctly claimed in the concluding portion of the specification. The present invention, however, both as to organization and method of operation, together with objects, features, and advantages thereof, may best be understood by reference to the following detailed description when read with the accompanying drawings in which:

FIG. 1 depicts solubility (mg/ml) of aspacytarabine with (i) Tween 20, 1% (non-linear polymer); (ii) PEG 400 (neat); (iii) an aqueous solution including 1% w/w poloxamer; (iv) an aqueous solution including 2% w/w poloxamer; and (v) an aqueous solution including 1% w/w poloxamer and 5% w/w PVP, where “P188” is poloxamer 188 and “PVP” is polyvinylpyrrolidone. The solubility was measured after 24 hours in room temperature at pH 4.5.

FIGS. 2A-2B depict stability of a dry composition comprising aspacytarabine and poloxamer 188 at weight ratios of 77:23 (FIG. 2A) or 10:1 (FIG. 2B).

FIG. 3 depicts solubility of aspacytarabine in an aqueous solution including 20% w/w of different kinds of cyclodextrins (after 24 hours at room temperature at pH 4.5) and 10% w/w of aspacytarabine.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

Aspacytarabine Pharmaceutical Composition

According to one aspect, the present invention provides a pharmaceutical composition comprising (S)-2-amino-4-((1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (Aspacytarabine) or a pharmaceutically acceptable salt thereof and at least one water soluble stabilizer and/or solubilizer.

According to one aspect, the present invention provides a pharmaceutical composition comprising (S)-2-amino-4-((1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (Aspacytarabine) or a pharmaceutically acceptable salt thereof and at least one water soluble stabilizer and/or solubilizer, wherein the at least one water soluble stabilizer and/or solubilizer is selected from a linear polymer, an inclusion compound and combination thereof. In another embodiment, the inclusion compound is cyclodextrin.

In one embodiment, the salt is selected from the group consisting of acetate salt, hydrochloride, hydrobromide salt, TFA salt, methanesulfonate salt, phosphate salt, citrate salt, lactate salt, succinate salt, tartarate salt, borate salt, benzoate salt, toluenesulfonate salt, benzenesulfonate salt, ascorbate acid, bisulfate salt, sulfate salt, maleate salt, formate salt, malonate salt, nicotinate salt and oxalate salt. In one embodiment, the salt is a hydrochloride salt. Each possibility represents a separate embodiment of the invention.

According to one aspect, the present invention provides a pharmaceutical composition comprising (S)-2-amino-4-((1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (Aspacytarabine) free base and at least one water soluble stabilizer and/or solubilizer, wherein the at least one water soluble stabilizer and/or solubilizer is selected from a linear polymer, an inclusion compound and combination thereof. In another embodiment, the inclusion compound is cyclodextrin.

According to one aspect, the present invention provides a pharmaceutical composition comprising (S)-2-amino-4-((1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (Aspacytarabine) salt and at least one water soluble stabilizer and/or solubilizer, wherein the at least one water soluble stabilizer and/or solubilizer is selected from a linear polymer, an inclusion compound and combination thereof. In another embodiment, the inclusion compound is cyclodextrin.

According to some embodiments of the present invention, the weight ratio between the aspacytarabine and the water soluble stabilizer and/or solubilizer is between 99:1 and 1:10. In one embodiment, the ratio is between 99:1 to 99:9. In another embodiment, the ratio is between 99:9 to 99:49. In another embodiment, the ratio is between 99:49 to 1:1. In another embodiment, the ratio is between 1:2 to 1:5. In another embodiment, the ratio is between 1:5 to 1:10. Each possibility represents a separate embodiment of the present invention. In another embodiment, the weight ratio is between 80:20 and 60:40. In another embodiment, the weight ratio is between 40:60 and 20:80. In another embodiment, the weight ratio is between 30:70 and 10:90.

In another embodiment, the weight percentage of aspacytarabine or its pharmaceutically acceptable salt is between 1% and 99%, relative to the total weight of the composition. In another embodiment, the weight percentage of aspacytarabine or its pharmaceutically acceptable salt is between 75% and 95%, relative to the total weight of the composition. In another embodiment, the weight percentage of aspacytarabine or its pharmaceutically acceptable salt is between 50% and 80%, relative to the total weight of the composition. In another embodiment, the weight percentage of aspacytarabine or its pharmaceutically acceptable salt is between 10% and 80%, relative to the total weight of the composition. In another embodiment, the weight percentage of aspacytarabine or its pharmaceutically acceptable salt is between 10% and 50%, relative to the total weight of the composition. In another embodiment, the weight percentage of aspacytarabine or its pharmaceutically acceptable salt is between 10% and 30%, relative to the total weight of the composition. In another embodiment, the weight percentage of aspacytarabine or its pharmaceutically acceptable salt is between 5% and 15%, relative to the total weight of the composition. In another embodiment, the weight percentage of aspacytarabine or its pharmaceutically acceptable salt is between 1% and 10%, relative to the total weight of the composition. Each possibility represents a separate embodiment of the invention.

In one embodiment, the weight percentage of the at least one water soluble linear polymer and/or inclusion compound is between 0.1 and 30% w/w relative to the total weight of the composition. In another embodiment, the weight percentage is between 0.1 and 0.5% w/w relative to the total weight of the composition. In another embodiment, the weight percentage is between 0.5 and 1% w/w relative to the total weight of the composition. In another embodiment, the weight percentage is between 1 and 2% w/w relative to the total weight of the composition. In another embodiment, the weight percentage is between 2 and 5% w/w relative to the total weight of the composition. In another embodiment, the weight percentage is between 5 and 10% w/w relative to the total weight of the composition. In another embodiment, the weight percentage is between 10 and 20% w/w relative to the total weight of the composition. In another embodiment, the weight percentage is 1 or 3% w/w relative to the total weight of the composition. Each possibility represents a separate embodiment of the invention. In another embodiment, the inclusion compound is cyclodextrin.

According to some embodiments of the present invention, the at least one water soluble stabilizer and/or solubilizer is a water soluble linear polymer. In another embodiment, the linear polymer is ionic or non-ionic. In some embodiments, non-ionic water soluble linear polymer comprise poly(vinyl alcohol), polyacrylamide, polyethylene glycol (polyethylene oxide) (PEG), polyethylene oxide (PEO) or polyoxyethylene (POE), triblock copolymers comprising polyoxypropylene (poly(propylene oxide)) and two polyoxyethylene (poly(ethylene oxide)) (poloxamer), polyvinyl pyrrolidone (PVP), derivative thereof or any combination thereof. In some embodiments, ionic water soluble linear polymer comprise ionic derivatives of poly(vinyl alcohol), polyacrylamide, polyethylene glycol (polyethylene oxide) (PEG), polyethylene oxide (PEO) or polyoxyethylene (POE), triblock copolymers comprising polyoxypropylene (poly(propylene oxide)) and two polyoxyethylene (poly(ethylene oxide)) (poloxamer), polyvinyl pyrrolidone (PVP), polyaspartic acid (PAA), Polysuccinimide (PSI), polystyrene sulfonic acid, polystyrene sulfonates derivatives thereof or any combination thereof.

In another embodiment, the at least one water soluble linear polymer is poloxamer. In another embodiment, the at least one water soluble linear polymer is combination of poloxamer and polyvinyl pyrrolidone (PVP). Each possibility represents a separate embodiment of the present invention.

In another embodiment, the composition of this invention comprises aspacytarabine and poloxamer. In another embodiment, the composition comprises aspacytarabine and a combination of poloxamer and polyvinyl pyrrolidone (PVP). In another embodiment, the composition comprises aspacytarabine and cyclodextrin. Each possibility represents a separate embodiment of the present invention.

According to some embodiments of the present invention, the at least one water soluble stabilizer and/or solubilizer is an inclusion compound. The term “inclusion compound” refers to a compound which acts as a “host” having a cavity where the aspacytarabine is accommodated as a “guest”. In some embodiments, the inclusion compound is a calixarene, cyclodextrin or combination thereof. In another embodiment, the inclusion compound is calixarene. In another embodiment, the inclusion compound is cyclodextrin. In another embodiment, the at least one water soluble stabilizer and/or solubilizer is cyclodextrin. In another embodiment, non-limiting examples of cyclodextrin (CD) include α-CD, β-CD, γ-CD, HP-β-CD (hydroxypropylated), SBE-β-CD (sulfobutyl-ether—modified), RM-β-CD (randomly methylated) or any combination thereof.

According to some embodiments of the present invention, the composition is chemically and physically stable when stored at a temperature of between −80 to 30° C. for at least one month or for at least for 24 hrs as an aqueous solution.

“Chemical stability” is herein defined as stability due to inertness of aspacytarabine compound within the composition. Thus, high chemical stability means reduced propensity of aspacytarabine compound to react/decompose/degrade over time.

“Physical stability” is herein defined as stability of a composition due to reduced possibility of changes in the physical, macro (visible) structure of the composition. Thus, “high physical stability” means for example a clear aqueous solution of the composition of this invention followed by suspension with time.

The term “water soluble stabilizer” refers to a chemical ingredient which is water soluble that stabilizes the aspacytarabine free base compound or pharmaceutically acceptable salt thereof and prevents its decomposition/degradation and preserves the chemical and physical stability of the composition and the active ingredients. In some embodiments, the water soluble stabilizer is also a solubilizer. In some embodiments, the water soluble stabilizer is selected from a water soluble linear polymer, an inclusion compound or combination thereof. In another embodiment, the inclusion compound is cyclodextrin.

The term “water soluble solubilizer” refers to a chemical ingredient which is soluble in water and solubilize the aspacytarabine “free base compound” or pharmaceutically acceptable salt thereof in an aqueous phase. In some embodiments, the water soluble solubilizer is also a stabilizer.

In some embodiments, the water soluble solubilizer is selected from a water soluble linear polymer, an inclusion compound or combination thereof. In another embodiment, the inclusion compound is cyclodextrin.

In one embodiment, the storage temperature range for the composition of this invention or an aqueous solution comprising thereof is between −80 to 30° C. In another embodiment, the storage temperature range for the composition of this invention or an aqueous solution comprising thereof is between 15-30° C. In another embodiment, the storage temperature range for the composition of this invention or an aqueous solution comprising thereof is between 25-30° C. In another embodiment, the storage temperature range for the composition of this invention or an aqueous solution comprising thereof is between 2-8° C. In another embodiment, the storage temperature range for the composition of this invention or an aqueous solution comprising thereof is between 8-15° C. In another embodiment, the storage temperature range for the composition of this invention or an aqueous solution comprising thereof is between 0-15° C. In another embodiment, the storage temperature range for the composition of this invention or an aqueous solution comprising thereof is between 0-10° C. In another embodiment, the storage temperature range for the composition of this invention or an aqueous solution comprising thereof is between 0-20° C. In another embodiment, the storage temperature range for the composition of this invention or an aqueous solution comprising thereof is between 0-30° C. In another embodiment, the storage temperature range for the composition of this invention or an aqueous solution comprising thereof is between −80 to 10° C. Each possibility represents a separate embodiment of the present invention.

In one embodiment, the composition is stable at the temperature ranges discussed above (i.e. −80° C. to 30° C.) for at least 1 month. In another embodiment, the composition is stable for at least 2 months. In another embodiment, the composition is stable for at least 3 months. In another embodiment, the composition is stable for at least 4 months. In another embodiment, the composition is stable for at least 5 months. In another embodiment, the composition is stable for at least 6 months. In another embodiment, the composition is stable for at least 7 months. In another embodiment, the composition is stable for at least 8 months. In another embodiment, the composition is stable for at least 9 months. In another embodiment, the composition is stable for at least 10 months. In another embodiment, the composition is stable for at least 11 months. In another embodiment, the composition is stable for at least one year. In another embodiment, the composition is stable for between 1 and 3 months. In another embodiment, the composition is stable for between 3 and 6 months. In another embodiment, the composition is stable for between 6 and 9 months. In another embodiment, the composition is stable for between 6 and 24 months. In another embodiment, the composition is stable for between 12 and 24 months. In another embodiment, the composition is a dry composition. In another embodiment, the composition is an aqueous solution. Each possibility represents a separate embodiment of the present invention.

In one embodiment, the composition is formulated as an aqueous solution and is stable for at least 24 hrs as an aqueous solution at −80° C. to 30° C. In another embodiment, the aqueous solution is stable for between 24 hrs to 36 hrs at −80° C. to 30° C. In another embodiment, the aqueous solution is stable for between 24 hrs to 48 hrs at −80° C. to 30° C. In another embodiment, the aqueous solution is stable for between 24 hrs to 72 hrs at 80° C. to 30° C. In another embodiment, the aqueous solution is stable for between 24 hrs up to a week at 80° C. to 30° C..

According to some embodiments of the present invention, the solubility of the aspacytarabine within the pharmaceutical composition as described herein in aqueous solution is at least 20 mg/ml. According to some embodiments of the present invention, the solubility of the aspacytarabine within the pharmaceutical composition as described herein in aqueous solution is between 20- 1000 mg/ml. In one embodiment, the solubility is between 20-200 mg/ml. In one embodiment, the solubility is between 20-500 mg/ml. In one embodiment, the solubility is between 20-200 mg/ml. In one embodiment, the solubility is between 20-50 mg/ml. In another embodiment, the solubility is between 50-75 mg/ml. In another embodiment, the solubility is between 75-100 mg/ml. In another embodiment, the solubility is between 100-125 mg/ml. In another embodiment, the solubility is between 125-150 mg/ml. In another embodiment, the solubility is between 150-200 mg/ml. In another embodiment, the solubility is between 150-300 mg/ml. In another embodiment, the solubility is between 250-500 mg/ml. Each possibility represents a separate embodiment of the present invention. In another embodiment, the solubility is between 150-500 mg/ml. In another embodiment, the solubility is between 200-750 mg/ml. In another embodiment, the solubility is between 500-1000 mg/ml. In another embodiment, the solubility is between 150-1000 mg/ml. Each possibility represents a separate embodiment of the present invention.

According to some embodiments of the present invention, the concentration of the aspacytarabine within the pharmaceutical composition as described herein in aqueous solution is at least 20 mg/ml. According to some embodiments of the present invention, the concentration of the aspacytarabine within the pharmaceutical composition as described herein in aqueous solution is between 20- 1000 mg/ml. In one embodiment, the concentration is between 20-200 mg/ml. In one embodiment, the concentration is between 20-500 mg/ml. In one embodiment, the concentration is between 20-200 mg/ml. In one embodiment, the concentration is between 20-50 mg/ml. In another embodiment, the concentration is between 50-75 mg/ml. In another embodiment, the concentration is between 75-100 mg/ml. In another embodiment, the concentration is between 100-125 mg/ml. In another embodiment, the concentration is between 125-150 mg/ml. In another embodiment, the concentration is between 150-200 mg/ml. In another embodiment, the concentration is between 150-300 mg/ml. In another embodiment, the concentration is between 250-500 mg/ml. In another embodiment, the concentration is between 150-500 mg/ml. In another embodiment, the concentration is between 200-750 mg/ml. In another embodiment, the concentration is between 500-1000 mg/ml. In another embodiment, the concentration is between 150-1000 mg/ml. Each possibility represents a separate embodiment of the present invention.

According to some embodiments of the present invention, the composition is formulated as a parenteral, oral, intranasal or inhalation composition. In one embodiment, the parenteral composition is selected from a solution, a suspension, an emulsion for injection or infusion, particles for injection or infusion, liposomes as injectable delivery system, a powder for injection or infusion, and a gel for injection. In another embodiment, the parenteral composition is administered by intravenous, intraarterial, intramuscular, subcutaneous, intraperitoneal, intracerebral, intracerebroventricular, intrathecal or intradermal administration route. In another embodiment, the oral composition is selected from a tablet, a pill, a capsule, a dragé, a gel, a syrup, a slurry, a suspension, a powder, or a liquid form. Each possibility represents a separate embodiment of the present invention.

According to some embodiments of the present invention, the composition further comprises a pharmaceutically acceptable carrier. In one embodiment, the carrier is water, saline solution, isotonic solution, aqueous dextrose, multiple electrolyte injection or aqueous glycerol solution. Each possibility represents a separate embodiment of the present invention.

According to some embodiments of the present invention, the composition is formulated for infusion or injection in a pharmaceutically acceptable carrier, wherein the carrier is selected from water, saline solution, isotonic solution, solutions accepted for infusion, aqueous dextrose or aqueous glycerol solution, wherein the composition having a pH range of between 2.2 and 8. In one embodiment, the pH range is between 4 and 8. In another embodiment, the pH range is between 7 and 8. In another embodiment, the pH range is between 4-5. In another embodiment, the pH is physiological. In another embodiment, a buffer is used in order to maintain and/or adjust the required pH range. In another embodiment, the buffer can be a pharmaceutically acceptable mono-ionic buffer system or a poly-ionic buffer system having an ionization pK in the range of 2.2-8. In another embodiment, various buffers can be used, for example, ACES (N-(acetamido)-2-aminoethansulfonic acid); Acetate; N-(2-acetamido)-2-iminodiacetic acid; BES (N,N-bis[2-hydroxyethyl]-2-aminoethansulfonic acid); Bicine (2-(Bis(2-hydroxyethyl)amino)acetic acid); Bis-Tris methane (2-[Bis(2-hydroxyethyl)amino]-2-(hydroxymethyl)propane-1,3-diol); Bis-Tris propane (1,3-bis(tris(hydroxymethyl)methylamino)propane); Carbonate; Citrate; 3,3-dimethyl glutarate; DIPSO (3-[N,N-bis(2-hydroxyethyl)amino]-2-hydroxypropansulfonic acid); N-ethylmorpholine; Glycerol-2-phosphate; Glycine; Glycine-amid; HEPBS (N-(2-hydroxyethyl) piperazin-N′-4-buthanesulfonic acid); HEPES (N-(2-hydroxyethyl)piperazin-N′-2-ethanesulfonic acid); HEPPS (N-(2-hydroxyethyl)piperazin-N′-(3-propanesulfonic acid)); HEPPSO (N-(2-hydroxyethyl)piperazin-N′-(2-hydroxypropanesulfonic acid); Histidine; Hydrazine; Imidazole; Maleate; 2-methylimidazole; MES (2-(N-morpholino)ethanesulfonic acid); MOBS (4-(N-morpholino)-butansulfonic acid); MOPS (3-(N-morpholino)-propanesulfonic acid; MOPSO (3-(N-morpholino)-2-hydroypropanesulfonic acid); Oxalate; Phosphate; Piperazine; PIPES (1,4-Piperazine-diethanesulfonic acid); POPSO (Piperazine-N,N′-bis(2-hydroxypropanesulfonic acid)); Succinate; Sulfite; TAPS (3-[[1,3-dihydroxy-2-(hydroxymethyl) propan-2-yl]amino]propane-1-sulfonic acid); TAPSO (3-[[1,3-dihydroxy-2-(hydroxymethyl) propan-2-yl]amino]-2-hydroxypropane-1-sulfonic acid); Tartaric acid; TES (2-[[1,3-dihydroxy-2-(hydroxymethyl) propan-2-yl]amino]ethanesulfonic acid); THAM (Tris) (2-Amino-2-hydroxymethyl-propane-1,3-diol); and Tricine (N-(2-Hydroxy-1,1-bis(hydroxymethyl)ethyl)glycine); a carboxylic acid derivative buffer including, but not limited to, Acetatate, N-(2-acetamido)-2-iminodiacetic acid, 2-(Bis(2-hydroxyethyl)amino)acetic acid, Carbonate, Citrate, 3,3-dimethyl glutarate, Lactate, Maleate, Oxalate, Succinate, and Tartaric acid buffer; an amino acid derivative buffer including, but not limited to, Bicine, Glycine, Glycine-amid, Histidine, and Tricine buffer; a phosphoric acid derivative buffer including, but not limited to, Glycerol-2-phosphate and phosphate buffer; and other buffer systems such as: Hank's balanced salt solution, Earle's balanced salt solution, Gey's balanced salt solution, HEPES buffered saline, phosphate buffered saline, Plasma-lyte, Ringer's solution, Ringer Acetate, Ringer lactate, Saline citrate, Tris buffered saline, acid-citrate-dextrose solution and Elliott's B solution; and any combination thereof. Each possibility represents a separate embodiment of the present invention.

In other embodiments, pharmaceutical compositions for parenteral administration include aqueous solutions of the active ingredients in water-soluble form. Additionally, suspensions of the active compound may be prepared as appropriate oily injection suspensions. Suitable lipophilic solvents or vehicles include fatty oils such as sesame oil, or synthetic fatty acids esters such as ethyl oleate, triglycerides or liposomes.

In other embodiment, the composition of this invention may be formulated as a liquid formulation. In another embodiment, the composition is a dry composition.

Without wishing to be bound to any theory or mechanism of action, aspacytarabine, an amino-acid-cytarabine conjugate of the composition of the present invention, is transported into the cancer cells and within the cells these conjugates are cleaved to release cytarabine which arrests cell growth or kill the cell. As free cytarabine and free cytarabine metabolites were detected in cancer cells, the conjugates of the present invention act as pro-drugs. These pro-drugs are stabilized and/or dissolved due to the at least one water soluble linear polymer, inclusion compound or combination thereof employed herein in the compositions of the present invention. In another embodiment, the inclusion compound is cyclodextrin.

The compositions can be formulated as solutions, suspensions, emulsion, tablets, pills, capsules, powders, sustained-release formulations and the like. The composition can be formulated as a suppository, with traditional binders and carriers such as triglycerides, microcrystalline cellulose, gum tragacanth or gelatin. Each possibility represents a separate embodiment of the present invention.

The composition can further comprise excipients including, but not limited to, sodium chloride, potassium chloride, magnesium chloride, sodium gluconate, sodium acetate, calcium chloride, sodium lactate, and the like. The composition, if desired, can also contain minor amounts of sugar alcohols, wetting or emulsifying agents, and pH adjusting agents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. Each possibility represents a separate embodiment of the present invention.

For oral administration, the composition of this invention can be formulated readily by combining aspacytarabine or pharmaceutical acceptable salt thereof and at least one water soluble stabilizer and/or solubilizer selected from a linear polymer, an inclusion compound or combination thereof with additional components as known in the art. Such components enable the composition of the invention to be formulated as tablets, pills, dragees, capsules, liquids, gels, syrups, slurries, suspensions, and the like, for oral ingestion by a subject. Pharmacological preparations for oral use can be made using a solid components, optionally grinding the resulting mixture, and processing the mixture of granules, after adding suitable auxiliaries if desired, to obtain tablets or dragee cores. Suitable components are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose. If desired, disintegrating agents may be added, such as cross-linked polyvinyl pyrrolidone, agar or alginic acid or a salt thereof such as sodium alginate. In another embodiment, the inclusion compound is cyclodextrin.

In addition, enteric coating can be useful if it is desirable to prevent exposure of the compounds of the invention to the gastric environment.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added.

For buccal administration, the compositions may take the form of tablets or lozenges formulated in conventional manner.

For administration by inhalation, the active compound for use according to the present invention are conveniently delivered in the form of an aerosol spray presentation from a pressurized pack or a nebulizer with the use of a suitable propellant, e. g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e. g., gelatin for use in an inhaler or insufflator may be formulated containing a powder mix of the peptide and a suitable powder base such as lactose or starch.

An intranasal composition may be formulated as a powder, an aqueous solution or a non-aqueous solution. A preferred method of administering the solutions of the invention is using a spray device. Spray devices can be single (“unit”) dose or multiple dose systems. The powder formulation is preferably administered to the patient in aerosolized form whereby energy from patient inhalation (sniffing) is used to aerosolize the powder into the nasal cavity or where the device itself provides the aerosolization energy, such as via compressed air.

Process of Preparing Aspacytarabine Composition

According to one further aspect, the present invention provides a process of preparing a composition as described hereinabove, comprising aspacytarabine or a pharmaceutically acceptable salt thereof and at least one water soluble stabilizer and/or solubilizer the process comprises:
mixing aspacytarabine or a pharmaceutically acceptable salt thereof and at least one water soluble stabilizer and/or solubilizer in an aqueous or organic solvent or mixtures thereof in a ratio of the aspacytarabine or a pharmaceutically acceptable salt thereof and the at least one water soluble stabilizer and/or solubilizer being between 99:1 and 1:10;
adjusting the pH of the mixture to pH of between 2.2 and 8 if an aqueous mixture is used; and
optionally drying the composition to obtain a dry composition.

In another embodiment, the dry composition is further formulated to parenteral, oral, intranasal or inhalation composition.

In one embodiment, the organic solvent comprises ethanol, methanol, propylene glycol, dimethylacetamide (DMA), polyethylene glycols (PEG).

According to some embodiments of the present invention, the mixing step is conducted at temperature of 2-30° C. In one embodiment, the temperature is between 2-8° C. In another embodiment, the temperature is between 2-15° C. In another embodiment, the temperature is between 8-15° C. In another embodiment, the temperature is between 8-25° C. In another embodiment, the temperature is between 15-25° C. Each possibility represents a separate embodiment of the present invention.

According to some embodiments of the present invention, the pH in the process of preparing the aspacytarabine composition, is adjusted to between 2.2 and 8. In one embodiment, of the present invention, the pH is adjusted to between 4 and 8. In another embodiment, the pH is adjusted to between 4-5. In another embodiment, the pH is physiological. Each possibility represents a separate embodiment of the present invention.

According to some embodiments of the present invention, the drying of the adjusted pH solution is done using any method known in the art, for example lyophilization, vacuum drying, heating and any combination thereof.

Therapeutic Use

According to one additional aspect, the present invention provides a method of treating a neoplastic disease comprising administering to a subject in need of such treatment a composition as described herein above.

In one embodiment, the subject is a medically compromised subject who is not amenable to treatment with high or standard doses of an anti-cancer agent, e.g., cytarabine. The medically compromised subject may be selected from the group consisting of elderly subjects, subjects having hepatic dysfunction, subjects having renal dysfunction, subjects having pancreatic dysfunction, subjects having bone marrow dysfunction, subjects having cerebellar dysfunction, subjects having immunological disorder, subjects having refractory or relapsed hematological cancer, and any combination thereof.

According to some embodiments of the present invention, the neoplastic disease is selected from the group consisting of hematological cancers and non-hematological cancers. In another embodiment, the hematological cancer is selected from the group consisting of leukemias, lymphomas, myelomas and Myelodysplastic Syndromes (MDS). In one embodiment, leukemia is selected from the group consisting of Acute Myeloid Leukemia (AML), Acute Lymphoblastic Leukemia (ALL), Chronic Myeloid Leukemia (CML), and Chronic Lymphoblastic Leukemia (CLL). In another embodiment, the AML is selected from the group consisting of newly diagnosed AML, secondary AML, and relapsed/refractory AML. In another embodiment, the lymphoma is selected from the group consisting of Hodgkin's lymphoma and non-Hodgkin's lymphoma. Each possibility represents a separate embodiment of the present invention.

According to some embodiments of the present invention, the composition is administered parenterally, orally or by inhalation. In one embodiment, the composition is administered by intravenous (i.v.), intraarterial, intramuscular, subcutaneous, intraperitoneal (i.p.), intracerebral, intracerebroventricular, intrathecal or intradermal administration route. In another embodiment, the composition is administered at a daily dose wherein the aspacytarabine dosage is ranging from about 0.3 g/m² to about 10 g/m² of the subject's surface area, for a period of at least 3 days. In another embodiment, the dosage is ranging from about 0.3 g/m² to about 1 g/m². In another embodiment, the dosage is ranging from about 1 g/m² to about 2 g/m². In another embodiment, the dosage is ranging from about 2 g/m² to about 5 g/m². In another embodiment, the dosage is ranging from about 2.5 g/m² to about 10 g/m². In another embodiment, the dosage is ranging from about 3 g/m² to about 10 g/m². In another embodiment, the dosage is ranging from about 5 g/m² to about 10 g/m². In another embodiment, the period is of at least 4 days. In another embodiment, the period is of at least 5 days. In another embodiment, the period is of at least 6 days. In another embodiment, the period is of at least 7 days. In another embodiment, the period is of at least 10 days. In another embodiment, the composition is administered by intravenous infusion for a period ranging from 15 minutes to 24 hours. In another embodiment, the composition is administered by intravenous infusion for a period ranging from 15 minutes to 0.5 hours. In another embodiment, the composition is administered by intravenous infusion for a period ranging from 0.5 hour to 1 hour. In another embodiment, the composition is administered by intravenous infusion for a period ranging from 1 hour to 3 hours. Each possibility represents a separate embodiment of the present invention.

According to a certain embodiment, the pharmaceutical composition is administered by intravenous infusion for 30 minutes to 24 hours. The compositions of the invention may be administered locally and may further comprise an additional active agent and/or excipient.

According to further embodiments, the compound of the invention is administered in a daily dosage of at least 1.5, 2, 3, 5, 10, 15, 20, or at least 30 times greater than the standard of care dose of cytarabine. Each possibility represents a separate embodiment of the invention.

According to some embodiments, the composition of the present invention is administered at least once a month until the subject reaches a remission. According to additional embodiments, the composition is administered at least twice a month until the subject reaches a remission. According to further embodiments, the composition is administered at least once a week until the subject reaches a remission. According to yet further embodiments, the composition is administered at least twice a week until the subject reaches a remission. According to still further embodiments, the composition is administered once a day for at least one week or until the subject reaches a remission. According to further embodiments, the composition is administered at least once a day for at least one week or until the subject reaches a remission.

According to some embodiments, the composition is administered once a day for at least 2, 3, 4, 5, 6, 8, 10, 12, or at least 14 consecutive days once a month. Alternatively, the composition is administered once a day for at least 2, 3, 4, 5, 6, or 12 days, or further alternatively the composition is administered every day or twice a week until the patient reaches a remission.

The composition can also be delivered by slow-release delivery systems, pumps, and other known delivery systems for continuous infusion. Dosing regimens may be varied to provide the desired circulating levels of a particular compound based on its pharmacokinetics. Thus, doses are calculated so that the desired circulating level of a therapeutic agent is maintained.

Typically, the effective dose is determined by the activity and efficacy of the compound and the condition of the subject as well as the body weight or surface area of the subject to be treated. The dose and the dosing regimen are also determined by the existence, nature, and extent of any adverse side effects that accompany the administration of the compounds in a particular subject.

Definitions

As used herein a “pharmaceutical composition” refers to a preparation of one or more of the compounds described herein, or physiologically acceptable salts or solvents thereof, with other chemical components such as physiologically suitable carriers and excipients. The purpose of a pharmaceutical composition is to facilitate administration of a compound to a subject.

The term “dry composition” refers herein to a composition of this invention which is not in an aqueous solution. In another embodiment, a “dry composition” refers to a composition comprising aspacytarabine and at least one water soluble stabilizer and/or solubilizer which followed a drying step. The “dry composition” can be further used for the preparation of any formulation (liquid, aqueous solutions, parenteral, oral, intranasal or inhalation composition)

The terms “renal dysfunction”, “hepatic dysfunction”, “pancreatic dysfunction”, “bone marrow dysfunction” and “cerebellar dysfunction” refer to a state in which the organ/tissue function, e.g., kidney, liver, pancreas, bone marrow, and cerebellum, is decreased relative to a normal state. In general, organ/tissue dysfunction is a state characterized in that any one or more measurement values of inspection items for organ function are deviated from the range of normal values (reference values).

The terms “standard of care dose” and “the recommended maximal dose” of cytarabine are used herein interchangeably and refer to the dosage, e.g., the daily dose, of cytarabine approved by the U.S. FDA for administration to a human subject, which dosage does not cause unacceptable adverse effects and is dependent on the subject's age and physical condition so that a fit subject of 70 or less years of age can be typically treated with a daily dose of cytarabine of up to 3 g/m² (Standard dose is between 100 to 400 mg/m²) a subject of 75 or more years of age can be treated with a daily dose of cytarabine of up to 20 mg/m² of the subject's surface area. However, it should be noted that most of the subjects of 75 or more years of age cannot be treated with cytarabine at all due to its severe adverse effects.

The terms “treatment”, “treat”, “treating” and the like, are meant to include slowing, arresting or reversing the progression of a disease. These terms also include alleviating, ameliorating, attenuating, eliminating, or reducing one or more symptoms of a disease, even if the disease is not actually eliminated and even if progression of the disease is not itself slowed or reversed. A subject refers to a mammal, preferably a human being.

The term “about” in reference to a numerical value stated herein is to be understood as the stated value +/−10%.

The term “pharmaceutically acceptable salt” of a drug refers to a salt according to IUPAC conventions. Pharmaceutically acceptable salt is an inactive ingredient in a salt form combined with a drug. Typical pharmaceutically acceptable salts include those salts prepared by reaction of the compounds of the present invention with a pharmaceutically acceptable mineral, base, acid or salt. Acid salts are also known as acid addition salts (see herein below). Pharmaceutically acceptable salts are known in the art (Stahl and Wermuth, 2011, Handbook of pharmaceutical salts,

Second edition). The acid is selected from the group consisting of acetic acid, hydrochloric acid, hydrobromic acid, methanesulfonic acid, phosphoric acid, citric acid, lactic acid, succinic acid, tartaric acid, boric acid, benzoic acid, toluenesulfonic acid, benzenesulfonic acid, ascorbic acid, sulfuric acid, bisulfuric acid, maleic acid, formic acid, malonic acid, nicotinic acid, oxalic acid and trifluoroacetic acid. In one embodiment, the salt is a hydrochloride salt. Each possibility represents a separate embodiment of the invention.

The term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents.

The following examples are to be considered merely as illustrative and non-limiting in nature. It will be apparent to one skilled in the art to which the present invention pertains that many modifications, permutations, and variations may be made without departing from the scope of the invention.

### EXAMPLE 1

### Aspacytarabine Dissolution Attempts

Aspacytarabine or its hydrochloride salt was stirred with various ingredients in various conditions (separate experiment for each component/condition) in order to prepare compositions thereof. Numerous compositions showed insufficient chemical stability (showing e.g. low purity or high decomposition percentages) or physical stability (the compositions were in a form of unstable suspensions, turbid solutions, precipitates in solutions etc.). Among components tested were organic solvents (e.g. glycerol, ethanol, polyethylene glycol (PEG, e.g. PEG 400), dimethylacetamide and propylene glycol) and polymers (e.g. Polyoxyethylated 12-hydroxystearic acid, Polyethylene glycol, sorbitan monolaurate, polyvinylpyrrolidone (PVP), triblock copolymer of polyethylene/propylene oxide (poloxamer) and cyclodextrin and/or derivatives thereof). The conditions tested included: various temperatures (4-30° C.), pH ranges, stirring durations, poloxamer, cyclodextrin (and derivatives thereof), PVP, polyethylene glycol or combinations thereof were found to stabilize and/or dissolve successfully the aspacytarabine. See comparative results in FIG. 1 and Table 1:

TABLE 1
Solubility of aspacytarabine in different solubilizers/stabilizers.

| Solubilizer/Stabilizer | Conc (mg/ml) |
|---|---|
| PEG400 | 50.49 |
| Poloxamer 1% | 95.26 |
| Poloxamer 2% | 94.94 |
| Poloxamer 1% + 5% PVP | 90.9 |
| Tween 20 | 14.33 |
| Solutol HS15 | 16.15 |
| Kolliphor ELP | 17.8 |

### EXAMPLE 2

### Compositions of this Invention

Composition Comprising Aspacytarabine and Poloxamer 188 (Composition 1)

A dry composition consisting of:

10% w/w aspacytarabine or pharmaceutically acceptable salt thereof;

0.5-5-% w/w poloxamer 188

0.1M NaOH to adjust pH.

The dry composition was stable for at least 12 months. The dry composition can be further dissolved in physiological aqueous solution for injection or infusion.

Composition comprising aspacytarabine and cyclodextrines

Composition 2a:

A composition of an aqueous solution consisting of:

10% w/w aspacytarabine or pharmaceutically acceptable salt thereof;

20-30-% w/w Captisol (SBEβCD); and

0.1M NaOH to adjust pH.

Composition 2b:

A composition of an aqueous solution consisting of:

10% w/w aspacytarabine or pharmaceutically acceptable salt thereof;

20-30-% w/w Kleptose HP (K-HP); and

0.1M NaOH to adjust pH.

Composition 2c:

A composition of an aqueous solution consisting of:

10% w/w aspacytarabine or pharmaceutically acceptable salt thereof;

20-30-% w/w Kleptose HPB (K-HPB); and

0.1M NaOH to adjust pH.

Composition 2d:

A composition of an aqueous solution consisting of:

10% w/w aspacytarabine or pharmaceutically acceptable salt thereof;

20-30-% w/w Kleptose HPB LB (K-HPB LB); and

0.1M NaOH to adjust pH.

Composition Comprising Aspacytarabine and Poloxamer 188 and PVP (Composition 3)

A composition consisting of:

10% w/w aspacytarabine or pharmaceutically acceptable salt thereof;

5-% w/w PVP;

1% w/w poloxamer 188; and

0.1M NaOH to adjust pH.

Composition Comprising Aspacytarabine and PEG400 (Composition 4)

A composition consisting of:

10% w/w aspacytarabine or pharmaceutically acceptable salt thereof;

90-% w/w PEG 400.

### EXAMPLE 3

### Stability and Solubility of Compositions of the Present Invention

Compositions 1, 2a-2d, 3 as described in Example 2 were dissolved in an aqueous solution at room temperature. Composition 4 is aspacytarabine in neat PEG 400. Solubility of aspacytarabine was measured after 24 hours and represented results are presented in FIG. 1. As can be seen, the solubility of the composition comprising poloxamer 188, PEG and combination of poloxamer and PVP demonstrated high solubility.

In another study, aspacytarabine hydrochloride salt was added to 3% aqueous solution of different stabilizers/solubilizers (See Table 2) at pH˜5. Solubility and stability of the solutions were tested following 24 hours in room temperature, as can be seen in Table 1.

TABLE 2
Aspacytarabine stability in different stabilizers

| | | | Initial | Concentration |
|---|---|---|---|---|
| | Initial | Appearance | concentration, | after 24 h, |
| Stabilizers/Solubilizers | appearance | after 24 h | mg/mL | mg/mL |
| Poloxamer 188 | Clear | Clear | 87.3 ± 0.2 | 81.8 ± 3.2 |
| Poloxamer 188 + | Clear | Clear | 87.3 ± 0.4 | 80.6 ± 2.2 |
| PEG 300 | | | | |
| PVP 10 kDa + | Clear | Slightly | 87.1 ± 0.0 | 91.6 ± 8.4 |
| PEG 300 | | turbid | | |
| Control without | Clear | Heavy | 86.6 ± 1.1 | 17.2 ± 0.4 |
| Stabilizers and/or | | precipitation | | |
| solubilizer | | | | |

Accordingly, the compositions of this invention comprising aspacytarabine or pharmaceutically acceptable salt thereof and poloxamer, PEG, PVP, or combination thereof, provides stable solutions with a solubility of above 50mg/ml

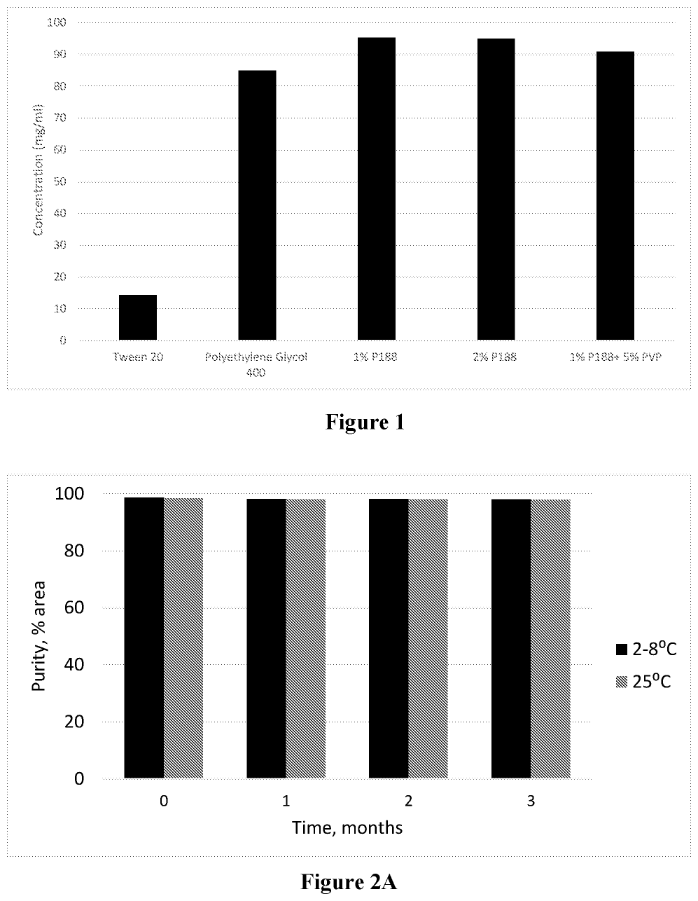

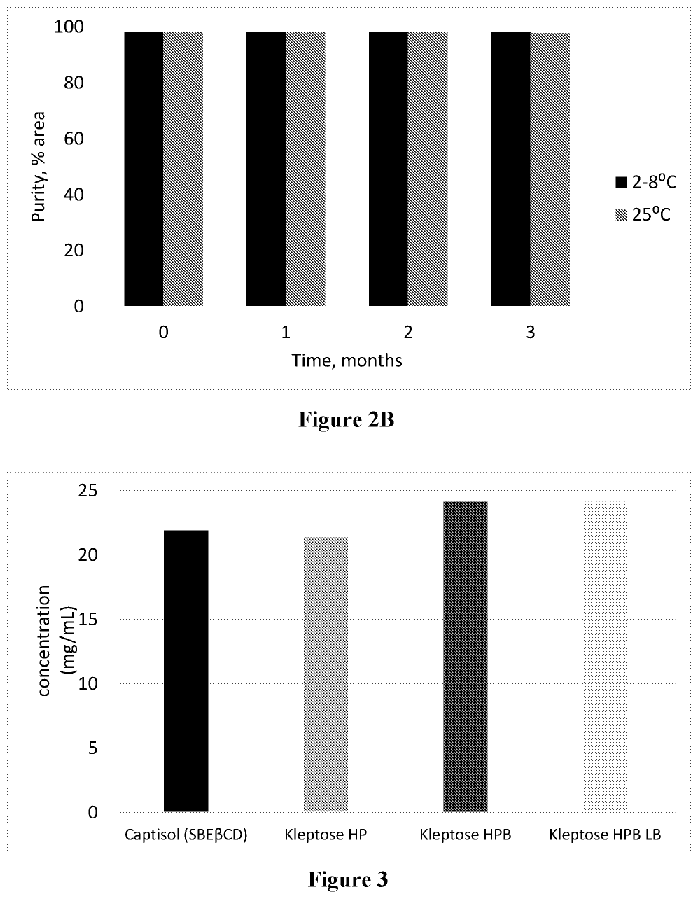

In a third study, aspacytarabine or pharmaceutically acceptable salt and poloxamer 188 at a weight ratio of 77:23 (a) or 10:1 (b) were mixed, the pH was adjusted to 4.5 and the resulting solutions were freeze dried. Stability of the dried compositions were tested over time, as illustrated in FIGS. 2A (77:23 ratio) - 2B (10:1 ratio).

In a final study, aspacytarabine solubility was measured in presence of 20% w/w of different cyclodextrins after 24 hours at room temperature at pH 4.5, and the results are shown in FIG. 3.

In view of the presented results, it is shown that the compositions of the present invention are stable over time in terms of the aspacytarabine concentration (solubility) and purity and the solutions appearance. This stability and solubility of aspacytarabine within the compositions of the present invention is due to the use of least one water soluble linear polymer, cyclodextrin or combination thereof (see for example Table 2 and FIGS. 1-3).

It is appreciated by persons skilled in the art that the present invention is not limited by what has been particularly shown and described hereinabove. Rather the scope of the present invention includes both combinations and sub-combinations of various features described hereinabove as well as variations and modifications. Therefore, the invention is not to be constructed as restricted to the particularly described embodiments, and the scope and concept of the invention will be more readily understood by references to the claims, which follow.

1.-31. (canceled)

32. A composition comprising (S)-2-amino-4-((1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (aspacytarabine) or pharmaceutically acceptable salt thereof and at least one water soluble stabilizer and/or solubilizer.

33. The composition of claim 32, wherein the at least one water soluble stabilizer and/or solubilizer is a linear polymer, an inclusion compound or combination thereof.

34. The composition according to claim 33, wherein the at least one water soluble linear polymer is non-ionic or ionic.

35. The composition according to claim 33, wherein said water soluble linear polymer comprises poly(vinyl alcohol), polyacrylamide, polyethylene glycol (polyethylene oxide) (PEG), polyethylene oxide (PEO) or polyoxyethylene (POE), triblock copolymers comprising polyoxypropylene (poly(propylene oxide)) and two polyoxyethylene (poly(ethylene oxide)) (poloxamer), polyvinyl pyrrolidone (PVP) or combination thereof.

36. The composition according to claim 32, wherein the composition comprises aspacytarabine and poloxamer or aspacytarabine and a combination of poloxamer and polyvinyl pyrrolidone (PVP).

37. The composition according to claim 32 wherein the composition comprises aspacytarabine and cyclodextrin.

38. The composition according to claim 33, wherein the weight ratio between the aspacytarabine and the linear polymer or the inclusion compound is between 99:1 to 1:10.

39. The composition according to claim 33, wherein the at least one water soluble linear polymer or the inclusion compound is at a weight percentage of between 0.1% and 30% w/w.

40. The composition according to claim 32, wherein the composition is formulated as a parenteral, oral, intranasal or inhalation composition.

41. The composition according claim 32, wherein the composition is formulated for infusion or injection in an aqueous media, wherein the aqueous media is at pH range of between 4 and 8.

42. The composition of claim 32, wherein the aspacytarabine salt is a hydrochloride salt.

43. A method of treating a neoplastic disease comprising administering to a subject in need of such treatment a composition according to claim 32.

44. The method according to claim 43, wherein the composition is administered parenterally, orally or by inhalation.

45. The method of claim 43, wherein the composition is administered at a daily dose wherein the aspacytarabine dosage is ranging from about 0.3 g/m2 to about 10 g/m2 of the subject's surface area, for a period of at least 3 days.

46. A composition comprising (S)-2-amino-4-((1-((2R,3S,4S,5R)-3,4-dihydroxy-5-(hydroxymethyl) tetrahydrofuran-2-yl)-2-oxo-1,2-dihydropyrimidin-4-yl)amino)-4-oxobutanoic acid (aspacytarabine) or pharmaceutically acceptable salt thereof and at least one water soluble stabilizer and/or solubilizer; wherein the composition is chemically and physically stable when stored at a temperature between −80 to 30° C. for at least 1 month and for at least 24 hrs when formulated as an aqueous solution.

47. The composition according to claim 46, wherein the at least one water soluble stabilizer and/or solubilizer is a linear polymer, an inclusion compound or combination thereof.

48. The composition according to claim 47, wherein the at least one water soluble linear polymer is non-ionic or ionic.

49. The composition according to claim 47, wherein said water soluble linear polymer comprises poly(vinyl alcohol), polyacrylamide, polyethylene glycol (polyethylene oxide) (PEG), polyethylene oxide (PEO) or polyoxyethylene (POE), triblock copolymers comprising polyoxypropylene (poly(propylene oxide)) and two polyoxyethylene (poly(ethylene oxide)) (poloxamer), polyvinyl pyrrolidone (PVP) or combination thereof.

50. The composition according to claim 46, wherein the composition comprises aspacytarabine and poloxamer or aspacytarabine and a combination of poloxamer and polyvinyl pyrrolidone (PVP).

51. The composition according to claim 46 wherein the composition comprises aspacytarabine and cyclodextrin.

52. The composition according to claim 47, wherein the weight ratio between the aspacytarabine and the linear polymer or the inclusion compound is between 99:1 to 1:10.

53. The composition according to claim 47, wherein the at least one water soluble linear polymer or the inclusion compound is at a weight percentage of between 0.1% and 30% w/w.

54. The composition according to claim 46, wherein the composition is formulated as a parenteral, oral, intranasal or inhalation composition.

55. The composition according to claim 46, wherein the composition is formulated for infusion or injection in an aqueous media, wherein the aqueous media is at pH range of between 4 and 8.

56. The composition of claim 46, wherein the aspacytarabine salt is a hydrochloride salt.

57. A method of treating a neoplastic disease comprising administering to a subject in need of such treatment a composition according to claim 46.

58. The method according to claim 57, wherein the composition is administered parenterally, orally or by inhalation.

59. The method of claim 57, wherein the composition is administered at a daily dose wherein the aspacytarabine dosage is ranging from about 0.3 g/m2 to about 10 g/m2 of the subject's surface area, for a period of at least 3 days.

60. A composition comprising (S)-2-amino-4-((1-((2R,3S,4S,5R)-3,4-dihydroxy (hydroxymethyl)tetrahydrofuran-2-yl)-2-oxo-1,2-dihydropyrimidin-4-yl)amino) oxobutanoic acid (aspacytarabine) or pharmaceutically acceptable salt thereof and at least one water soluble stabilizer and/or solubilizer wherein the concentration of the aspacytarabine within the composition in aqueous solution is between 20 -1000 mg/ml.

61. The composition of claim 60, wherein the at least one water soluble stabilizer and/or solubilizer is a linear polymer, an inclusion compound or combination thereof.

62. The composition according to claim 61, wherein the at least one water soluble linear polymer is non-ionic or ionic.

63. The composition according to claim 61, wherein said water soluble linear polymer comprises poly(vinyl alcohol), polyacrylamide, polyethylene glycol (polyethylene oxide) (PEG), polyethylene oxide (PEO) or polyoxyethylene (POE), triblock copolymers comprising polyoxypropylene (poly(propylene oxide)) and two polyoxyethylene (poly(ethylene oxide)) (poloxamer), polyvinyl pyrrolidone (PVP) or combination thereof.

64. The composition according to claim 60, wherein the composition comprises aspacytarabine and poloxamer or aspacytarabine and a combination of poloxamer and polyvinyl pyrrolidone (PVP).

65. The composition according to claim 60 wherein the composition comprises aspacytarabine and cyclodextrin.

66. The composition according to claim 61, wherein the weight ratio between the aspacytarabine and the linear polymer or the inclusion compound is between 99:1 to 1:10.

67. The composition according to claim 61, wherein the at least one water soluble linear polymer or the inclusion compound is at a weight percentage of between 0.1% and 30% w/w.

68. The composition according to claim 60, wherein the composition is formulated as a parenteral, oral, intranasal or inhalation composition.

69. The composition according to claim 60, wherein the composition is formulated for infusion or injection in an aqueous media, wherein the aqueous media is at pH range of between 4 and 8.

70. The composition of claim 60, wherein the aspacytarabine salt is a hydrochloride salt.

71. A method of treating a neoplastic disease comprising administering to a subject in need of such treatment a composition according to claim 60.

72. The method according to claim 71, wherein the composition is administered parenterally, orally or by inhalation.

73. The method of claim 71, wherein the composition is administered at a daily dose wherein the aspacytarabine dosage is ranging from about 0.3 g/m2 to about 10 g/m2 of the subject's surface area, for a period of at least 3 days.
